(19) United States
(12) Patent Application Publication
(10) Pub. No.: US 2002/0128637 A1
(43) Pub. Date: Sep. 12, 2002

(54) Laser scalpel

(76) Inventors: Hans-Joachim von der Heide, Eisenstadt (AT); Kurt Franz Zanglein, Eisenstadt (AT); Egon Alzner, Salzburg (AT)

Correspondence Address: Stephen A. Soffen, DICKSTEIN SHAPIRO MORIN & OSHINSKY LLP, 2101 L Street NW, Washington, DC, 20037-1526, US

(21) Appl. No.: 10/084,317

(22) Filed: Feb. 28, 2002

Related U.S. Application Data
(63) Continuation of application No. PCT/AT00/00233, filed on Aug. 30, 2000.

(30) Foreign Application Priority Data
Aug. 31, 1999 (AT) A 1500/99

(51) Int. Cl. A61B 18/22

(52) U.S. Cl. 606/16

(57) ABSTRACT

A laser scalpel for cutting biological tissue. The laser scalpel includes a suctioning device with a suction channel which is arranged parallel to a laser fiber. The suction channel protrudes beyond the end of the laser fiber, and the protruding portion of the suction channel includes a suction opening in the wall of the suction channel facing the laser beam. The suction opening is thus oriented towards the laser beam which exits from the distal end of the laser fiber. This prevents the suction channel from becoming blocked.

The invention relates to a laser scalpel for cutting biological tissue comprising a suction device, wherein the suction device exhibits a suction channel comprising a suction opening, which suction channel is arranged in parallel to a laser fibre equipped with a distal end being free in the axial direction of the laser fibre, and wherein a laser beam emerges from the distal end of the laser fibre.

Laser scalpels and laser tools, respectively, of that kind have for some time been used in the field of ophthalmic surgery, in particular for treating grey cataracts.

In the so-called cataract therapy, the lenticular nucleus of the eye is broken up and drawn off by the aid of energy. In doing so, the energy is absorbed by water present in the lenticular nucleus and is transformed into heat, whereby the protein of the lens is destroyed and the lens disintegrates into fractions or partially liquefies, respectively. That liquidification process is also called phacoemulsification. The thus treated lens is removed from the eye by means of a suction device.

Up until recently, exclusively the ultrasonic phacoemulsification was used for this method of treatment. Thereby, an ultrasound source provides the energy necessary for liquidification.

However, that technique involves the disadvantages of a high depth of penetration of the energy and a large heat input, which may not only result in a liquidification of the lenticular nucleus but may also damage the tissue surrounding the lens. For inserting the appropriate tool into the eye, relatively large cuts are required, which involve an increased risk for the patient.

Laser scalpels do not have those disadvantages since, in their case, the energy, in the form of a laser light having a selected wavelength, i.e. a specific energy, is transported via a light-conducting fibre to the lenticular nucleus, where it is completely absorbed already at a low depth. Due to the tool-specific arrangement of the light guide and the suction device, larger cuts may be avoided and the risk of a rupture of the capsule may be reduced.

Similar to instruments for ultrasound use, laser scalpels generally consist of a manually operable handpiece and an exchangeable working tip to be placed upon the handpiece as well as of connections to appropriate washing and suction devices. The working tip comprises an adaptor for mounting the tip to the handpiece, a cannula for suction, in which a fibre for light transmission is laterally installed, as well as, optionally, a further channel for washing.

In contrast to the ultrasonic phacoemulsification, laser therapy may leave relatively solid residual matters due to the small energy supply; fragmentation of the lenticular nucleus rather than a liquidification of the same takes place. The residual matters left by the laser therapy as well as untreated fragments of the lenticular nucleus are drawn off by means of a suction system into which the light guide is integrated. Lacking volume is replaced by a suitable filling material, f.i., a salt solution, either via a separate wash handpiece or a wash cannula integrated into the laser-suction handpiece.

According to an embodiment, such as, f.i., U.S. Pat. No. 5,112,328 A, the laser scalpel working tips known in the art have an arrangment wherein a light-conducting fibre is fixed to the interior wall of the suction cannula and ends flush with the distal end of the cannula. The cannula's interior space not occupied by the fibre serves as a suction channel for the lens fragments.

However, especially in case of harder lenticular nuclei, the flush arrangement of the fibre and the suction opening causes plugging of the suction cannula, since fragments which have not been broken up sufficiently by the laser beam are drawn to the opening and may seal the same. The lacking separation between the fibre and the suction device and the geometry of the suction channel resulting therefrom also increase the tendency of blocking suction, particularly since drawn-off particles may get squeezed or stuck, respectively, in the vertically angular longitudinal edges between the concave interior wall of the suction channel and the convex surface of the laser beam.

Deficient suction of the lenticular nuclei is one of the main reasons why, so far, the laser therapy has been unable to find acceptance as opposed to the ultrasonic phacoemulsification. The fact that solid components, in particular of lenticular nuclei with greater density, plug the suction cannula leads to extremely long durations of treatment. The treatment must be interrupted several times, the handpiece must be removed from the eye to be washed, whereby the patient is exposed to an increased risk, in particular to an increased risk of infection.

From U.S. Pat. No. 4,694,828 A, a surgical laser device is known, with which tissue to be removed is evaporated by means of a laser beam which is generated within a chamber. The laser beam is recovered in an especially provided chamber opposite the distal end of the laser fibre, and the evaporated tissue is carried off via a suction channel. In such an embodiment, the distal end of the laser fibre is not free in the axial direction of the laser fibre for the purpose of protecting the surrounding tissue, but is covered by the above-described chamber.

From the documents U.S. Pat. No. 4,985,027 A, DE 38 31 141 A1 and DE 197 14 475 C1, laser scalpels of the initially described kind are known, in which the laser fibre is arranged within the suction channel, which means that merely tissue parts projecting through the suction opening into the interior of the suction channel can be treated.

From WO 91/06271 a surgical laser instrument is known, with which a pulsatory laser beam bounces against a transducer transforming the electromagnetic energy into mechanical shock waves which emerge from the surgical instrument through an opening of a suction channel.

The invention aims at avoiding the disadvantages and difficulties of the prior art, and its object is to provide a laser scalpel of the initially described kind which renders feasible undisturbed suction and, due to the thus shortened duration of treatment and a treatment to be carried out without any intermediate cleaning, substantially reduces the risk for the patient.

According to a first embodiment of the invention, that object is achieved in that the laser fibre is arranged outside the suction channel, that the suction channel rises above the distal end of the laser fibre, that the suction opening is provided in a wall directed towards the laser beam in the portion of the suction channel rising above the distal end of the laser fibre, that the suction opening is directed towards the laser beam emerging from the distal end of the laser fibre and that the suction channel has a rounded distal end.

A second embodiment is characterized in that the laser fibre is arranged outside the suction channel, that the suction channel rises above the distal end of the laser fibre, that the suction opening is provided in a wall directed towards the laser beam in the portion of the suction channel rising above the distal end of the laser fibre, that the suction opening is directed towards the laser beam emerging from the distal end of the laser fibre and that the laser scalpel has a jacket tube smooth on the outside.

According to a third embodiment, the laser fibre is arranged outside the suction channel, the suction channel rises above the distal end of the laser fibre, the suction opening is provided in a wall directed towards the laser beam in the portion of the suction channel rising above the distal end of the laser fibre, the suction opening is directed towards the laser beam emerging from the distal end of the laser fibre, and the suction channel exhibits a cross section which is constant throughout the length of the working tip.

If, during laser application, hard fragments of the lenticular nucleus and residual products occur, those are drawn in front of the fibre outlet area through the lateral suction opening. If the fragments are small enough, they are drawn off via the cannula. Otherwise, they are kept by suction in front of the fibre so that further smashing by means of laser is feasible. The fragments continue to be broken up until they can pass through the suction opening.

According to a previous but not pre-published publication WO 99/44554, a laser scalpel of the initially described kind is known, in which the laser fibre is indeed arranged outside the suction channel and the suction channel rises above the distal end of the laser fibre, whereby the suction opening is provided in a wall directed towards the laser beam in the portion of the suction channel rising above the distal end of the laser fibre, and the suction opening is directed towards the laser beam emerging from the distal end of the laser fibre, but the suction channel does not exhibit a rounded distal end but rather is designed with edges. Furthermore, that laser scalpel is not surrounded by a smooth jacket tube on the outside. And on the inside, the suction channel is designed conically throughout the length of the working tip.

According to the invention, the suction opening is preferably provided in a side wall rising above the distal end of the laser fibre and constituting the suction channel.

Preferably, the suction channel has a single suction opening in the side wall, whereby it is guaranteed that the lens fragments must in any case pass the fibre outlet area.

According to a preferred embodiment, the suction opening exhibits a smaller, preferably by at least 10% smaller, cross section than the suction channel. That guarantees that only fragments which are smaller than the cross section of the suction channel may get into the same. Thereby, plugging of the channel is ruled out.

Advantageously, the largest diameter of the suction opening is smaller than the smallest diameter of the suction channel.

Suitably, the sealed distal end of the suction channel is designed in a rounded fashion, reducing the risk of injuries caused by sharp edges during the insertion of the working tip into the eye.

A further preferred embodiment is characterized in that the laser scalpel has a tube in which, on one side, the laser fibre is arranged, and that, within the tube and opposite the laser fibre, the suction channel is designed to be partitioned off by a wall.

According to a further preferred embodiment, the suction channel is formed by a further tube being arranged within the tube and preferably having an elliptic cross section.

Advantageously, the laser scalpel is equipped with a further channel for supplying a filling material, such as, f.i., a salt solution etc. That has the advantage that no separate cut is necessary for washing, since the wash channel may be inserted while being integrated in a working tip together with the laser fibre and the suction device.

Thereby, the additional channel for supplying a filling material is suitably formed by a channel peripherally surrounding both the suction channel and the laser fibre.

With the suction opening seen in a front view, the suction opening is preferably covered for the most part, preferably completely, by the laser beam.

A suitable embodiment for a more manifold use of the laser scalpel is characterized in that the portion of the suction channel rising above the distal end of the laser fibre is roughened on the outside, with the roughness advantageously being in the range of from 20 to 60 μm, preferably from 25 to 50 μm.

In order to be able to carry out an opening of the capsule pouch in addition to the fragmentation of the lenticular nucleus, the normal line directed towards the outside of the wall of the suction channel, which wall is directed towards the laser beam and supports the suction opening, advantageously includes an angle of ≦90°, preferably an angle α of between 30° and 80°, with the longitudinal centre axis of the laser beam in the direction of the beam.

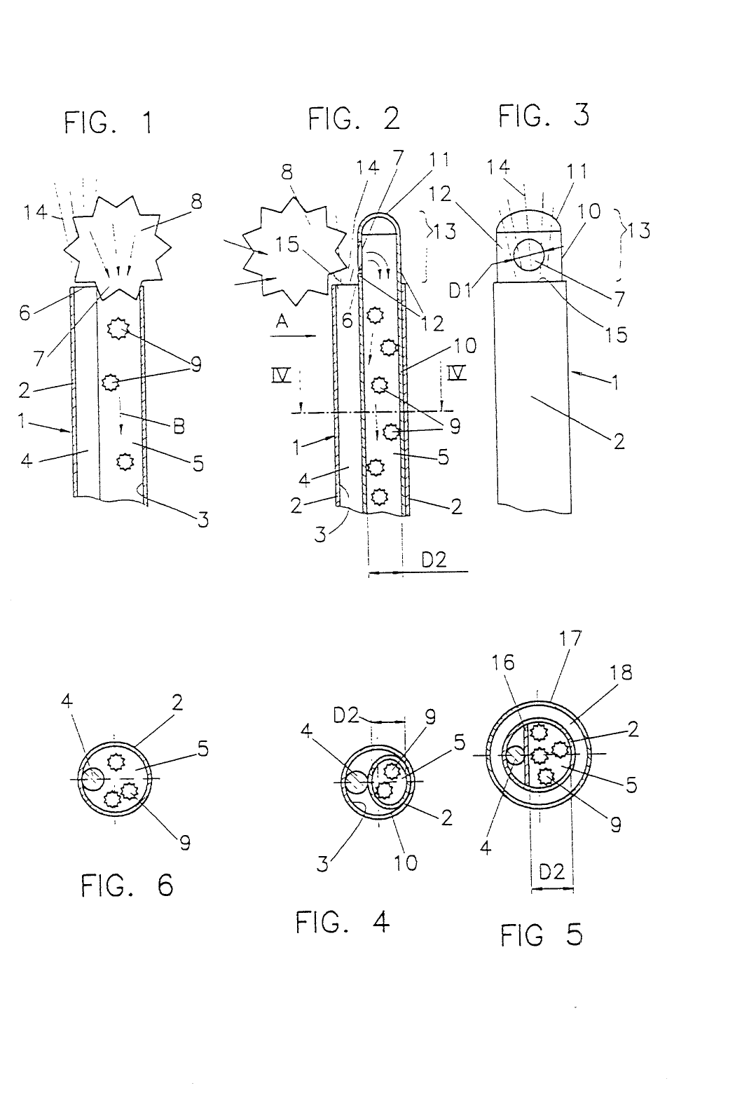

In the following, the invention is described in greater detail by the aid of the drawing, wherein FIG. 1 depicts a longitudinal section through a working tip of a prior art laser scalpel, FIG. 2 depicts a longitudinal section through a working tip of a laser scalpel of the invention, FIG. 3 depicts a top view according to arrow A of the laser scalpel working tip illustrated in FIG. 2, FIG. 4 depicts a section taken along line IV-IV through the laser scalpel working tip illustrated in FIG. 2, und FIG. 5 depicts a section comparable to FIG. 4 through another embodiment of a working tip of a laser scalpel of the invention.

The working tip 1 of a known laser scalpel is formed by a tube 2, called a jacket tube, to the inside 3 of which a laser fibre 4 is fixed, which laser fibre conducts the laser light necessary for the operation from the laser source to the operating area. The interior space not occupied by the laser fibre and formed by the jacket tube 2 serves as the suction channel 5, through which the fragmented lenticular nucleus is transported off by means of a suction device which is not illustrated. According to FIG. 1, the distal end 6 of the laser fibre 4 ends flush with the suction channel 5 or the jacket tube 2, respectively, i.e. the suction opening 7 is located on the same level as the distal end 6 of the laser fibre 4. If a particle 8 exhibiting a larger cross section than the suction opening 7 is drawn in, the particle 8 gets stuck in the suction opening 7 and prevents the smaller particles 9 from entering the suction channel 5, whereby suction is generally blocked and the laser scalpel has to be drawn out of the eye in order to be cleaned. (The arrows B in FIGS. 1 and 2 depict the flow direction of fragments 8 and 9).

The working tip 1 shown in FIG. 2 of a laser scalpel of the invention also has a jacket tube 2, f.i., with an outside diameter of 1.2 mm, with the jacket tube 2 being made of a material common in medicine, such as special steel.

A laser fibre 4 for use as a light guide in an infrared range of around 3 μm is fixed to the inside 3 of the jacket tube 2, with the proximal area of the laser fibre, the so-called main light guide (not shown), usually being made of zinc fluoride, whereas the distal area for bridging over the distance between the main light guide and the operating site is made of a conventional quartz fibre, since zinc fluoride is no biocompatible material. However, the quartz content of the light guide is kept as small as possible in order to minimize the attenuation of radiation caused by the quartz fibre. In this embodiment, the laser fibre 4 exhibits a diameter of about 200-300 μm. In accordance with the desired energy transfer, other diameters are also possible.

Opposite the laser fibre 4, a tube 10 having an elliptic cross section (see FIG. 4) is squeezed into the jacket tube 2 forming the suction channel 5. The cross section of the ellipse is dimensioned such that the jacket tube 2 is filled as best as possible.

The elliptic tube 10 rises above the end of the jacket tube 2 and the distal end 6 of the laser fibre 4, in the illustrated exemplary embodiment by about 500-600 μm, whereas the laser fibre 4 ends flush with the jacket tube 2. At its distal end 11, the tube 10 is sealed, with the distal end 11 of the tube 10 being designed in a rounded fashion.

In a side wall 12 of the protruding portion 13 of the tube 10, a suction opening 7 is provided, which is directed towards the laser beam 14 emerging from the distal end 6 of the laser fibre 4. However, the suction opening 7 could, for example, also be provided in a wall of the tube 10 tapering towards a rounded tip and being directed towards the laser beam 14, i.e., not in a right angle with the laser beam outlet area 15.

During operation, the fragments of the lenticular nucleus 8 and 9 are drawn in front of the suction opening 7 by the suction device, whereby they pass through the laser beam 14 and, optionally, are broken up by the same by means of a structurally enforced, continual contact with the laser beam 14 until they are small enough for getting into the tube 10 through the suction opening 7. Thereby, it is advantageous if the largest diameter D1 of the suction opening 7 is smaller than the smallest diameter D2 of the suction channel 5, which, in that exemplary embodiment, is the smallest diameter D2 of the elliptic tube 10.

In the representation of the view of the laser scalpel working tip 1 of FIG. 3, seen in the direction of arrow A of FIG. 2, the suction opening 7 is completely covered by the laser beam 14. The diameter D1 of the suction opening 7 has—as explained above—been chosen to be smaller than the small semiaxis of the elliptic tube 10, such as apparent from a comparison with FIG. 4.

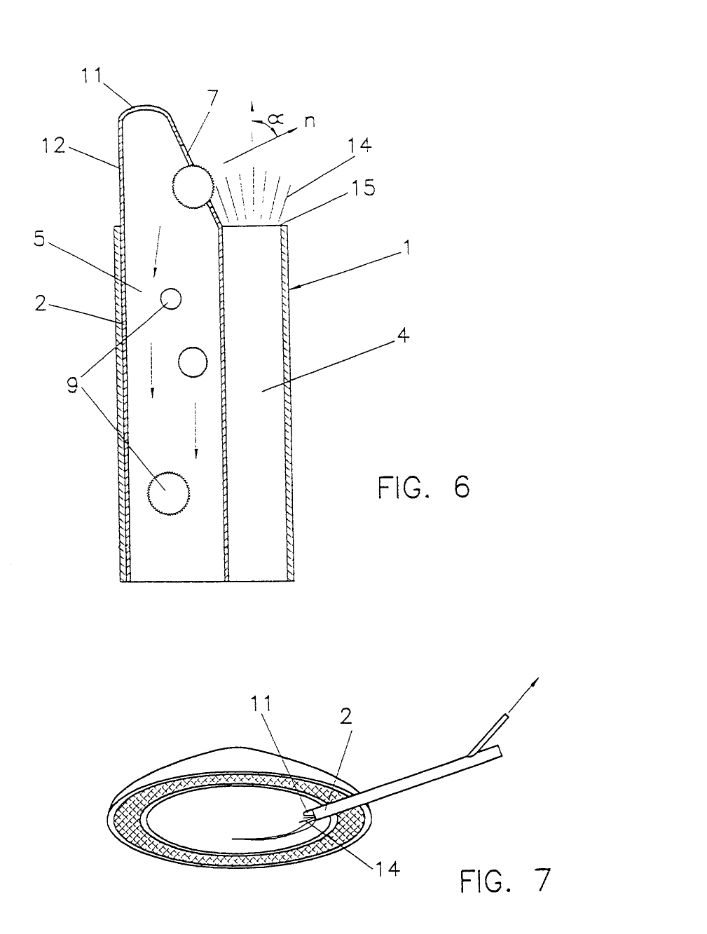

FIG. 4 illustrates a sectional view along line IV-IV of FIG. 2, with the separate suction channel 5 having an elliptic cross section being distinctly recognizable. Due to the arrangement according to the invention of the suction channel 5, there are no convex areas within the suction channel 5 which would favour plugging by particles 9 as those easily get jammed in the narrow recesses formed by the surfaces of the laser fibre 4 and the jacket tube 2. For that reason, the arrangement of the suction channel 5 and the laser fibre 4 in a jacket tube 2 as shown in FIG. 6 is a less preferred exemplary embodiment.

In FIG. 5, a further exemplary embodiment of a working tip 1 of a laser scalpel of the invention is depicted in greater detail, which, in particular, is suitable for laser fibres 4 having larger diameters. The sectional view shows a jacket tube 2 to the inside 3 of which a laser fibre 4 is fixed, with the laser fibre 4 being separated from a suction channel 5 formed by a portion of the jacket tube 2 by means of a wall 16. In this manner, it is still feasible to achieve satisfactory suction, with the jacket tube 2 having a cross section which is equal to the cross section of the jacket tube 2 of the embodiment illustrated in FIG. 2.

In this exemplary embodiment, the jacket tube 2 is coaxially surrounded by a further tube 17 forming an additional channel 18 for supplying a filling material and/or a washing liquid, which additional channel surrounds both the suction channel 5 and the laser fibre 4.

According to the embodiment illustrated in FIG. 6, the wall of the suction channel 5 supporting the suction opening 7 has a position which is inclined against the longitudinal direction of the laser scalpel, with the normal line n directed towards the outside of the wall of the suction channel 5, which wall is directed towards the laser beam 14 and supports the suction opening 7, including an angle α of between 30° and 80° with the longitudinal centre axis of the laser beam 14 in the direction of the beam.

By means of a laser scalpel of this embodiment, the opening of the capsule pouch., the so-called capsule orhexis (FIG. 7), may be effected in addition to the fragmentation of the lenticular nucleus. Thereby, the use of a special surgical instrument to this end is rendered superfluous.

Preferably, the portion of the suction channel 5 rising above the distal end of the laser fibre 4 is roughened on the outside, with the granulation being in the range of between 20 and 50 μm, preferably between 25 and 50 μm.

Upon completion of phacoemulsification, a thus designed working tip may be used for polishing the lens capsule prior to inserting the intra-ocular lens. The advantage over conventional laser scalpels is caused by the fact that a change of instruments between the two steps of treatment may be omitted, thereby reducing the risks of injury and infection for the patient.

The laser scalpel according to the invention is not limited to an application during cataract therapy; using the laser scalpel of the invention might also be conveivable, fi., for surgical interventions affecting the cartilaginous tissue.

1. Laser scalpel for cutting biological tissue comprising a suction device, wherein the suction device exhibits a suction channel (5) comprising a suction opening (7), which suction channel is arranged in parallel to a laser fibre (4) equipped with a distal end (6) being free in the axial direction of the laser fibre (4), wherein a laser beam (14) emerges from the distal end (6) of the laser fibre (4), the laser fibre (4) is arranged outside the suction channel (5), the suction channel (5) rises above the distal end (6) of the laser fibre (4), the suction opening (7) is provided in a wall (12) directed towards the laser beam (14) in the portion (13) of the suction channel (5) rising above the distal end (6) of the laser fibre (4), the suction opening (7) is directed towards the laser beam (14) emerging from the distal end (6) of the laser fibre (4) and the laser scalpel has a jacket tube (2) smooth on the outside, characterized in that the suction opening (7) is arranged in a distance from the distal end (6) of the laser fibre (4) in the axial direction of the suction channel (5).

2. Laser scalpel according to claim 1, characterized in that the suction channel (5) has a rounded distal end.

3. Laser scalpel according to claim 1 or 2, characterized in that the laser fibre (4) is immediately adjacent to the outer wall of the suction channel (5).

4. Laser scalpel according to one or several of claims 1 to 3, characterized in that the suction channel (5) exhibits a cross section which is constant throughout the length of the working tip (1).

5. Laser scalpel according to one or several of claims 1 to 4, characterized in that the suction opening (7) exhibits a circular cross section.

6. Laser scalpel according to one or several of claims 1 to 5, characterized in that the suction opening (7) is provided in a side wall (12) forming the suction channel (5), which side wall rises above the distal end (6) of the laser fibre (4).

7. Laser scalpel according to one or several of claims 1 to 6, characterized in that the suction channel (5) exhibits a single suction opening (7) in the side wall (12).

8. Laser scalpel according to one or several of claims 1 to 7, characterized in that the suction opening (7) exhibits a smaller, preferably by at least 10% smaller, cross section than the suction channel (5).

9. Laser scalpel according to one or several of claims 1 to 8, characterized in that the suction opening (7) exhibits a largest diameter (D1) which is smaller than the smallest diameter (D2) of the suction channel (5).

10. Laser scalpel according to one or several of claims 1 to 9, characterized in that, on one side of the jacket tube (2), the laser fibre (4) is arranged and that, within the jacket tube (2) and opposite the laser fibre (4), the suction channel (5) is designed as partitioned off by a wall (16).

11. Laser scalpel according to one or several of claims 1 to 10, characterized in that the suction channel (5) is formed by a further tube (10) being arranged within the jacket tube (2) and preferably having an elliptic cross section.

12. Laser scalpel according to one or several of claims 1 to 11, characterized in that the laser scalpel is equipped with a further channel (18) for supplying a filling material, such as, fi., a salt solution etc.

13. Laser scalpel according to claim 12, characterized in that the additional channel (18) for supplying a filling material is formed by a channel peripherally surrounding the jacket tube (2).

14. Laser scalpel according to one or several of claims 1 to 13, characterized in that, with the suction opening (7) seen in a front view, the suction opening (7) is covered for the most part, preferably completely, by the laser beam (14).

15. Laser scalpel according to one or several of claims 1 to 14, characterized in that the portion of the suction channel (5) rising above the distal end of the laser fibre (4) is roughened on the outside.

16. Laser scalpel according to claim 15, characterized in that the roughness is in the range of from 20 to 60 μm, preferably from 25 to 50 μm.

17. Laser scalpel according to one or several of claims 1 to 16, characterized in that the normal line (n) directed towards the outside of the wall of the suction channel (5), which wall is directed towards the laser beam (14) and supports the suction opening (7), includes an angle of ≦90°, preferably an angle α of between 30° and 80°, with the longitudinal centre axis of the laser beam (14) in the direction of the beam.
